(19) United States
(12) Patent Application Publication
(10) Pub. No.: US 2006/0135665 A1
(43) Pub. Date: Jun. 22, 2006

(54) Ester mixtures

(76) Inventors: Jan-Gerd Hansel, Koln (DE); Eberhard Kuckert, Leverkusen (DE); Johannes Kaulen, Odenthal (DE); Thomas Facklam, Leverkusen (DE)

Correspondence Address: LANXESS CORPORATION, 111 RIDC PARK WEST DRIVE, PITTSBURGH, PA, 15275-1112, US

(21) Appl. No.: 11/301,772

(22) Filed: Dec. 13, 2005

(30) Foreign Application Priority Data
Dec. 14, 2004 (DE) 10 2004 060 042.2

(51) Int. Cl. C08K 5/12; C07C 69/76

(52) U.S. Cl. 524/298; 560/90

(57) ABSTRACT

Ester mixtures comprising a mixture of esterification products formed from polyether polyols, one or more aromatic monocarboxylic acids or aromatic monocarboxylic acid derivatives and one or more aliphatic monocarboxylic acids or aliphatic monocarboxylic acid derivatives, to a process for their preparation, and to their use as plasticizers for polymers.

The present invention relates to ester mixtures comprising a mixture of esterification products formed from polyether polyols, one or more aromatic monocarboxylic acids or aromatic monocarboxylic acid derivatives and one or more aliphatic monocarboxylic acids or aliphatic monocarboxylic acid derivatives, to a process for their preparation, and to their use as plasticizers for polymers.

### BACKGROUND OF THE INVENTION

Plasticizers are substances which are added to brittle and hard polymers, for example polyvinyl chloride (PVC), in order to impart to them properties desirable for processing and use, such as flexibility and tensility.

The important substance properties of plasticizers for the application are described, for example, in David F. Cadogan, Christopher J. Howick: “Plasticizers”, Ullmann's Encyclopedia of Industrial Chemistry, Electronic Release, 6th ed., chap. 1-6, Wiley-VCH, Weinheim 2003 and L. Meier: “Plasticizers”, in R. Gächter, H. Müller (Ed.): Taschenbuch der Kunststoffadditive [Handbook of plastics additives], 3rd edition, p. 357-p. 382, Hanser Verlag, Munich 1990. In general, liquid plasticizers are used. They preferably have a viscosity of below 100 000 mPa·s, a dissolution temperature in polyvinyl chloride of below 170° C. and an acid number of below 1 mg KOH/g.

For the use of a substance as a plasticizer, it is also important that it remains substantially permanently within the polymer plasticized with it. Many plasticizers tend to migrate into substances in contact with the plasticized polymer, for example other polymers. Lubricants or blowing agents and also soap solutions can leach out plasticizers. Plasticizers with insufficient compatibility with the polymer to be plasticized can be deposited on the surface after processing and form an undesired, greasy film. Finally, plasticizers, owing to their volatility, can evaporate out of a plasticized polymer formulation. This leads firstly to undesired embrittlement of the polymer formulation and secondly to the likewise undesired precipitation of the plasticizer on cold surfaces. All of these phenomena occur to a particular degree when an object produced from plasticized plastic is exposed to elevated temperatures for a prolonged period. One example of these so-called high-temperature applications is that of cable sheathing which is used in the engine compartment of an automobile.

Preference is thus given to using plasticizers having low volatility, good compatibility and low migration tendency.

In addition to phosphoric esters and sulphonic esters, especially the alkyl esters of carboxylic acids have favourable substance properties for use as plasticizers. The technically relevant plasticizers and their use are known and are described, for example, in David F. Cadogan, Christopher J. Howick: “Plasticizers”, Ullmann's Encyclopedia of Industrial Chemistry, Electronic Release, 6th ed., chap. 1-6, Wiley-VCH, Weinheim 2003 and L. Meier: “Plasticizers”, in R. Gächter, H. Müller (Ed.): Taschenbuch der Kunststoffadditive, 3rd edition, p. 341 ff., Hanser, Munich 1990. The volatility of substances generally decreases with increasing molecular weight. Therefore, the esters used are usually not simple esters, but rather, owing to their higher molecular weights, preferably esters of polybasic carboxylic acids with monohydric alcohols, esters of monobasic carboxylic acids with polyhydric alcohols or esters of polybasic carboxylic acids with polyhydric alcohols. The latter group of esters comprises oligomeric or polymeric esters which, owing to their high viscosities, are more difficult to process than the former two groups of low molecular weight esters.

Esters of di- and tribasic carboxylic acids with monohydric alcohols are used the most frequently as plasticizers. Examples thereof are the phthalic esters, for example dioctyl phthalate (DOP), or trimellitic esters, for example trioctyl trimellitate (TOTM). Owing to their comparatively high volatility for high-temperature applications, diesters such as dioctyl phthalate are generally unsuitable. The corresponding triester of the tribasic trimellitic acid, trioctyl trimellitate, is established as a low-volatility plasticizer for high-temperature applications. However, trioctyl trimellitate is more difficult to process, less readily available and distinctly more expensive than dioctyl phthalate, and therefore cannot be used for many applications.

Some phthalic esters, for example dioctyl phthalate have in recent times come under suspicion of being harmful to health. Thus, according to the Dangerous Substances Directive 67/548/EEC, dioctyl phthalate has to be classified in the EU as possibly impairing fertility and possibly having developmental toxicity.

Esters of monobasic acids with dihydric alcohols can likewise be used advantageously as plasticizers. For example, U.S. Pat. No. 2,956,978 B1 describes dibenzoates of various glycols as plasticizers. As U.S. Pat. No. 6,184,278 B1 teaches, some of these dibenzoates, for example ethylene glycol dibenzoate, diethylene glycol dibenzoate or triethylene glycol dibenzoate, have the disadvantage that they are solid at 25° C.

U.S. Pat. No. 2,585,448 B1, U.S. Pat. No. 3,370,032 B1 and US 2003/0023112 A1 describe mixed esters which are prepared by reaction of diols with a mixture of aliphatic and aromatic carboxylic acids. However, the relatively high volatility of the diol esters excludes them from many applications.

DE 2 318 411 A1 proposes, as a plasticizer for hot-melt adhesive preparations, the substance trimethylolpropane tribenzoate. Since this substance is a solid, it is generally unsuitable as a plasticizer.

U.S. Pat. No. 3,072,591 B1 describes esters of polymethylolalkanes, for example trimethylolpropane, which contain, in the same molecule; both carboxyl radicals of at least one aromatic carboxylic acid and carboxyl radicals of aliphatic carboxylic acids of at least 6 carbon atoms. These so-called mixed esters are described as plasticizers for vinyl chloride polymers. However, their preparation requires a complicated two-stage preparation process (U.S. Pat. No. 3,072,591 B1, column 5, lines 15-17).

WO 02/053635 (US 2003 065073) describes mixtures of esters of trimethylolpropane, benzoic acid and 2-ethylhexanoic acid which are used as plasticizers, preferably for polyvinyl chloride. However, the use of ethylhexanoic acid is, according to the information of W. J. Scott, M. D. Collins, H. Nau; Environmental Health Supplements, Volume 102, Number S11, 1994, toxicologically controversial and should be avoided.

JP 06-25474 A describes plasticizers which are prepared by esterifying alkylene oxide adducts of polyhydric alcohols having 3 to 6 hydroxyl groups with aliphatic carboxylic acids which contain 8 to 24 carbon atoms. The plasticizers described in JP 06-25474 A are preferably used as plasticizers for rubber items. However, these plasticizers have a high dissolution temperature which makes them unsuitable for some applications (polyvinyl chloride).

JP 2003-171523 A describes plasticizers which are obtained by esterification reaction of an alkylene oxide addition product, of a polyhydric alcohol and of an aromatic carboxylic acid. The plasticizers described in JP 2003-171523 A are used exclusively as plasticizers for acrylate resins. However, these plasticizers are characterized by a high viscosity.

It is therefore an object of the present invention to provide plasticizers for polymers which can be prepared easily and have favourable processing properties, are liquid at room temperature and also remain liquid in the course of prolonged storage, feature low volatility and high thermal stability, and comprise a minimum of toxicologically controversial substances.

### SUMMARY OF THE INVENTION

This object is achieved by an ester mixture comprising at least two or more compounds of the general formula (I) 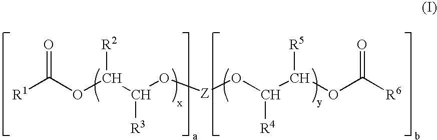
in which
R¹ is a straight-chain or branched C₅₋ to C₂₁-alkyl radical,
R^{2,} R³, R⁴ and R⁵ are each independently H or C₁₋ to C₄-alkyl radicals,
R⁶ is a C₆- to C₁₄-aryl radical which is optionally substituted by one to three C₁₋ to C₄₋alkyl radicals,
Z is a 3- to 6-valent aliphatic hydrocarbon radical,
x and y are each independently 0 to 6, where x+y has to be >0,
a is 0 to 6, where a has to be >0,
b is 0 to 6 and
   a is <or equals b and
   the sum of a+b equals the valency of the Z radical.

### DETAILED DESCRIPTION OF THE PREFERRED EMBODIMENTS OF THE INVENTION

The R¹ derives preferably from aliphatic monocarboxylic acids such as hexanoic acid, heptanoic acid, octanoic acid, nonanoic acid, decanoic acid, lauric acid, myristic acid, palmitic acid, stearic acid, oleic acid, arachic acid or behenic acid, especially from lauric acid.

The indices a, b, x and y specified in the formula (I) are based on the composition of the esters present in the inventive ester mixtures. This means either that the plasticizer can comprise esters in which all molecules are described by the same indices or that the plasticizer comprises an ester mixture in which the indices reproduce the different composition of all molecules.

The R⁶ radical derives preferably from aromatic monocarboxylic acids such as benzoic acid, o-toluic acid, m-toluic acid, p-toluic acid, 4-tert-butylbenzoic acid, 1-naphthoic acid or 2-naphthoic acid, in particular from benzoic acid.

The Z radical preferably corresponds to one of the general formulae (II) to (VI) 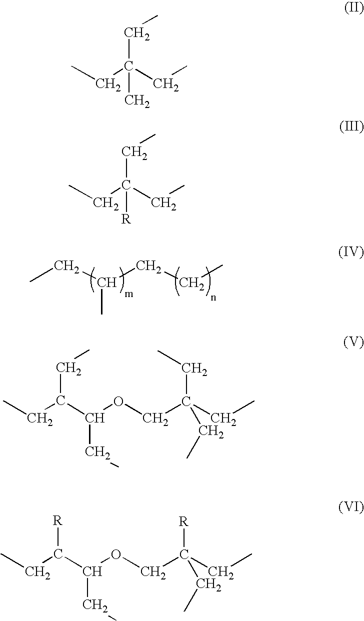
in which
R is H or a C₁₋ to C₄-alkyl chain,
m is 1 to 4 and
n is 0 to 3.

The Z radical preferably derives from low molecular weight aliphatic polyalcohols such as glycerol, diglycerol, 1,2,4-butanetriol, threitol, erythritol, trimethylolpropane, ditrimethylolpropane, trimethylolethane, pentaerythritol, dipentaerythritol, 1,2,6-hexanetriol, xylitol, mannitol, sorbitol or dulcitol, in particular from glycerol, trimethylolpropane or pentaerythritol. The Z radical particularly derives from trimethylolpropane.

The inventive esters typically have an acid number of less than or equal to 1 mg KOH/g. They preferably have an acid number of less than or equal to 0.5 mg KOH/g.

The invention also encompasses a process for preparing the, characterized in that

The invention also includes a process for preparing the ester mixture, characterized in that
a) one or more polyether polyols having in each case from 3 to 6 hydroxyl groups per molecule are esterified with
b) a mixture of
   1) at least 50 mol % of one or more aromatic C₇- to C₁₅-monocarboxylic acids or aromatic C₇- to C₁₅-monocarboxylic acid derivatives and
   2) at most 50 mol % of one or more aliphatic C₆- to C₂₂monocarboxylic acids or aliphatic C₆- to C₂₂-monocarboxylic acid derivatives
   where the mixtures 1) and 2) in total have to add up to 100%
c) optionally at elevated temperature,
d) optionally in the presence of catalysts and
e) optionally with removal of the water of reaction,
   it also being possible for the polyether polyols to be esterified in several steps with in each case only a portion of the total carboxylic acids to be used.

The molecular weight of the polyether polyols is typically between about 100 g/mol and about 2000 g/mol.

The polyether polyols are obtained preferably by ring-opening polymerization or copolymerization of one or more
cyclic aliphatic ethers such as ethylene oxide, propylene oxide, ethyloxirane, oxetan or tetrahydrofuran, especially ethylene oxide and/or propylene oxide and
   low molecular weight aliphatic polyalcohols having 3 to 6 hydroxyl groups in the molecule, such as glycerol, diglycerol, 1,-2-,4-butanetriol, threitol, erythritol, trimethylolpropane, ditrimethylolpropane, trimethylolethane, pentaerythritol, dipentaerythritol, 1,2,6-hexanetriol, xylitol, mannitol, sorbitol and/or dulcitol, especially glycerol, trimethylolpropane and/or pentaerythritol.

The esterification reactions can be accelerated with the aid of customary catalysts, for example titanium(IV) isopropoxide, titanium(IV) butoxide, tin(II) 2-ethylhexanoate, and/or of entraining agents, for example toluene or xylene. However, the inventive ester mixtures can also be prepared by the reaction of the above-described polyether polyols with derivatives of the carboxylic acids used, for example carboxylic esters, carboxylic anhydrides or carbonyl halides. These and further methods are known to those skilled in the art and are described, for example, in W. Riemenschneider: “Esters, Organic”, Ullmann's Encyclopedia of Industrial Chemistry, Electronic Release, 6th edition, ch. 5, Wiley-VCH, Weinheim 2003. In addition to the actual synthesis of the esters, their preparation can also include one or more workup steps, for example washing with water or aqueous solutions, bleaching, distillation, drying, filtration and the like.

The molar amount of all carboxylic acids used may be less than, equal to, or greater than the molar amount of the hydroxyl groups present in the polyether polyol used. The molar amount of all carboxylic acids used is preferably 80-150 mol % based on the molar amount of the hydroxyl groups present in the polyether polyol used. The molar amount of all carboxylic acids used is more preferably 90-120 mol % based on the molar amount of the hydroxyl groups present in the polyether polyol used.

After the end of the reaction, a residue of unconverted carboxylic acids may remain in the reaction mixture. This is to be expected in particular when more carboxylic acids are used than correspond to 100 mol % of the total of the hydroxyl groups present in the polyether polyol used. In the inventive preparation process, a residue of unconverted carboxylic acids is removed from the reaction mixture by one or more of the above-listed workup steps.

The aromatic C₇- to C₁₅-monocarboxylic acids or aromatic C₇- to C₁₅-monocarboxylic acid derivatives may preferably be unsubstituted or C₁- to C₄-alkyl-substituted.

The aliphatic C₆- to C₁₅-monocarboxyic acids or aliphatic C₆- to C₂₂-monocarboxylic acid derivatives may preferably be straight-chain or branched, saturated or olefinically unsaturated.

The cyclic aliphatic ethers used are preferably ethylene oxide and/or propylene oxide.

The polyether polyols used are preferably those which can be prepared from glycerol, trimethylolpropane or pentaerythritol. Very particular preference is given to using polyether polyols formed from trimethylolpropane which have a hydroxyl number between 100 and 1700 mg KOH/g.

By their nature, the polyether polyols are mixtures in which, in addition to polyethers of different chain length, the low molecular weight aliphatic polyalcohols used in the preparation reaction may also be present in portions. The molecular weights of the polyether polyols are average values. The polyether polyols used and their preparation are known and are described, for example, in G. Oertel: “Polyurethane”; in G. W. Becker, D. Braun (Ed.): Kunststoff-Handbuch [Plastics Handbook], 3rd Edition, Vol. 7, Carl Hanser-Verlag, Munich, 1992.

The aromatic C₇- to C₁₅-monocarboxylic acids or aromatic C₇- to C₁₅-monocarboxylic acid derivatives used are preferably benzoic acid, o-toluic acid, m-toluic acid, p-toluic acid, 4-tert-butylbenzoic acid, 1-naphthoic acid and/or 2-naphthoic acid, or derivatives of these acids or mixtures thereof. Preference is given to using benzoic acid.

The aliphatic C₆- to C₂₂-monocarboxylic acids or aliphatic C₆- to C₂₂-monocarboxylic acid derivatives used are preferably hexanoic acid, heptanoic acid, octanoic acid, nonanoic acid, decaenoic acid, lauric acid, myristic acid, palmitic acid, stearic acid, oleic acid, arachic acid and/or behenic acid, or derivatives of these acids or mixtures thereof. Preference is given to using lauric acid and/or palmitic acid.

Very particular preference is given to using
polyether polyols which can be prepared from trimethylolpropane and propylene oxide;
benzoic acid as the aromatic C₇- to C₁₅-monocarboxylic acid; and
lauric acid as the aliphatic C₆- to C₂₂-monocarboxylic acid.

Irrespective of the preparation method of the inventive esters, these may either be individual esters or mixtures of a plurality of esters. The inventive esters are mixtures especially when the polyether polyols used for their preparation are, as described, themselves mixtures of a plurality of components and/or when they have been prepared by using more than one carboxylic acid or more than one carboxylic acid derivative. In the case of ester mixtures, the acyl radicals of the carboxyl component or carboxyl components are distributed between the available hydroxyl functions of the alcohol component. This distribution may, for example, be random. As a consequence of the distribution of the acyl radicals, the ester mixture may contain individual ester molecules in which only the acyl radical of one of the carboxylic acids used is present, and also ester molecules in which two to six different acyl radicals are present.

The invention also encompasses the use of the ester mixtures as plasticizers for polymers such as polyvinyl chloride, vinyl chloride-based copolymers, polyvinylidene chloride, polyvinyl acetals, polyacrylates, polyamides, polylactides, cellulose and its derivatives, rubber polymers such as acrylonitrile-butadiene rubber, hydrogenated acrylonitrile-butadiene rubber, chloroprene rubber, chlorinated polyethylene, chlorosulphonyl polyethylene, ethylene-propylene rubber, acrylate rubber and/or epichlorohydrin rubber. Preference is given to polyvinyl chloride.

In this case, the polyvinyl chloride is prepared preferably by homopolymerization from vinyl chloride by methods known to those skilled in the art, such as suspension, emulsion or bulk polymerization. The inventive ester mixtures are used in mixtures with 20 to 99% polyvinyl chloride, preferably 45 to 95% polyvinyl chloride, more preferably 50 to 90% polyvinyl chloride. These mixtures are known as soft polyvinyl chloride and may, in addition to the inventive ester mixtures and polyvinyl chloride, also comprise other suitable additives. For example, stabilizers, lubricants, fillers, pigments, flame retardants, light stabilizers, blowing agents, polymeric processing assistants, impact modifiers; optical brighteners, antistats and/or biostabilizers may be present. All content data are percentages by weight.

The present invention also relates to polymers which comprise the inventive mixtures.

The polymers also preferably comprise additives such as stabilizers, lubricants, fillers, pigments, flame retardants, light stabilizers, blowing agents, polymeric processing assistants, impact modifiers, optical brighteners, antistats and/or biostabilizers, and also mixtures thereof.

Some suitable additives will be described in detail below. However, the examples adduced do not constitute any restriction of the inventive mixtures, but rather serve merely for illustration. All content data are % by weight.

Stabilizers neutralize the hydrochloric acid released during and/or after the processing of the polyvinyl chloride. Useful stabilizers are all customary polyvinyl chloride stabilizers in solid and liquid form, for example customary Ca/Zn, Ba/Zn, Pb or Sn stabilizers, and also acid-binding sheet silicates such as hydrotalcite. The inventive ester mixtures may be used in mixtures with a content of stabilizers of 0.05 to 7%, preferably 0.1 to 5%, more preferably 0.2 to 4% and in particular 0.5 to 3%.

Lubricants should be effective between the polyvinyl chloride particles and counteract frictional forces in the course of mixing, plasticizing and reshaping. The lubricants present in the inventive mixtures may be all lubricants which are customary for the processing of polymers. For example, useful lubricants are hydrocarbons such as oils, paraffins and PE waxes, fatty alcohols having 6 to 20 carbon atoms, ketones, carboxylic acids such as fatty acids and montanic acids, oxidized PE wax, metal salts of carboxylic acids, carboxamides and carboxylic esters, for example with the alcohols ethanol, fatty alcohols, glycerol, ethanediol, pentaerythritol and long-chain carboxylic acids as the acid component. The inventive ester mixtures may be used in mixtures having a content of lubricants of 0.01 to 10%, preferably 0.05 to 5%, more preferably 0.1 to 3% and in particular 0.2 to 2%.

Fillers influence in particular the compressive strength, tensile strength and flexural strength, and also the hardness and heat distortion resistance, of plasticized polyvinyl chloride or PVB in a positive way. In the context of the invention, the mixtures may also comprise fillers, for example carbon black and other inorganic fillers, such as natural calcium carbonates, for example chalk, limestone and marble, synthetic calcium carbonates, dolomite, silicates, silica, sand, diatomaceous earth, aluminium silicates, such as kaolin, mica and feldspar. The fillers used are preferably calcium carbonates, chalk, dolomite, kaolin, silicates, talc or carbon black. The inventive ester mixtures may be used in mixtures having a content of fillers of 0.01 to 80%, preferably 0.1 to 60%, more preferably 0.5 to 50% and in particular 1 to 40%.

The mixtures formulated with the inventive ester mixtures may also comprise pigments in order to adjust the resulting product to different possible uses. In the context of the present invention, both inorganic pigments and organic pigments may be used. The inorganic pigments used may, for example, be cadmium pigments such as CdS, cobalt pigments such as CoO/Al₂O₃, and chromium pigments, for example Cr₂O₃. The organic pigments used may, for example, be monoazo pigments, condensed azo pigments, azomethine pigments, anthraquinone pigments, quinacridones, phthalocyanine pigments, dioxazine pigments and aniline pigments. The inventive ester mixtures may be used in mixtures having a content of pigments of 0.01 to 10%, preferably 0.05 to 5%, more preferably 0.1 to 3% and in particular 0.5 to 2%.

In order to reduce the flammability and the evolution of smoke in the course of burning, the inventive mixtures may also comprise flame retardants. The flame retardants used may, for example, be antimony trioxide, phosphate esters, chloroparaffin, aluminium hydroxide, boron compounds, molybdenum trioxide, ferrocene, calcium carbonate or magnesium carbonate. The inventive ester mixtures may be used in mixtures having a content of flame retardant of 0.01 to 10%, preferably 0.1 to 8%, more preferably 0.2 to 5% and in particular 0.5 to 3%.

In order to protect articles which have been produced from a mixture comprising the inventive ester mixtures from damage in the surface region by the influence of light, the mixtures may also comprise light stabilizers. In the context of the present invention, it is possible, for example, to use hydroxybenzophenones or hydroxyphenylbenzotriazoles. The inventive ester mixtures may be used in mixtures having a content of light stabilizers of 0.01 to 7%, preferably 0.1 to 5%, more preferably 0.2 to 4% and in particular 0.5 to 3%.

The polymers may optionally comprise additional plasticizers from the group of monoalkyl esters of benzoic acid, benzoic diesters of mono-, di-, tri- or polyalkylene glycols, dialkyl esters of aliphatic diacids, dialkyl esters of aromatic diacids, trialkyl esters of aromatic triacids, phenyl esters of alkanesulphonic acids, alkyl or aryl esters of phosphoric acid, polyesters of dicarboxylic acids, and also mixtures thereof.

Examples of further plasticizers are
the monoalkyl esters of benzoic acid, for example isononyl benzoate,
the benzoic diesters of mono-, di-, tri- or polyalkylene glycols, for example propylene glycol dibenzoate, diethylene glycol dibenzoate, dipropylene glycol dibenzoate, triethylene glycol dibenzoate or polyethylene glycol dibenzoate,
the dialkyl esters of aliphatic diacids, for example di(2-ethylhexyl) adipate, diisononyl adipate, di(2-ethylhexyl) sebacate, di(2-ethylhexyl) azelate, diisononyl cyclohexane 1,2-dicarboxylate,
the dialkyl esters of aromatic diacids, for example di(2-ethylhexyl) phthalate, diisononyl phthalate, diisodecyl phthalate, benzyl butyl phthalate, benzyl isooctyl phthalate, benzyl isononyl phthalate,
the trialkyl esters of aromatic triacids, for example tri-(2-ethylhexyl) trimellitate,
the phenyl esters of alkanesulphonic acids, for example the product Mesamoll® from Lanxess Deutschland GmbH,
the alkyl or aryl esters of phosphoric acid, for example tri(2-ethylhexyl) phosphate, diphenyl 2-ethylhexyl phosphate, diphenyl cresyl phosphate or tricresyl phosphate,
polyesters which from dicarboxylic acids such as adipic acid or phthalic acid, and diols such as 1,2-propanediol, 1,3-butanediol, 1,4-butanediol or 1,6-hexanediol.

In the context of the invention, the inventive ester mixtures may also be used in mixtures which comprise further polymers selected from the group consisting of homo- and copolymers based on ethylene, propylene, butadiene, vinyl acetate, glycidyl acrylate, glycidyl methacrylate, acrylates and methacrylates having alcohol components of branched or unbranched C₁- to C₁₀-alcohols, styrene or acrylonitrile. Examples include polyacrylates having identical or different alcohol radicals from the group of the C₄- to C₈-alcohols, particularly of butanol, hexanol, octanol and 2-ethylhexanol, polymethyl methacrylate, methyl methacrylate-butyl acrylate copolymers, methyl methacrylate-butyl methacrylate copolymers, ethylene-vinyl acetate copolymers, chlorinated polyethylene, nitrile rubber, acrylonitrile-butadiene-styrene copolymers, ethylene-propylene copolymers, ethylene-propylene-diene copolymers, styrene-acrylonitrile copolymers, acrylonitrile-butadiene rubber, styrene-butadiene elastomers and methyl methacrylate-styrene-butadiene copolymers.

The mixtures prepared with the inventive ester mixtures are, for example, useful for the production of casings for electrical equipment, for example kitchen equipment and computer casings, pipelines, appliances, cables, wire sheathing, window profiles, in interior design, in vehicle and furniture construction, in floorcoverings, medical articles, food packaging, gaskets, films, composite films, films for composite safety glass, in particular for the vehicles sector and the architecture sector, phonographic disks, synthetic leather, toys, packaging containers, adhesive tape films, clothing, coatings, as fibres for fabrics.

The inventive ester mixtures have good processability and low volatility. Soft polyvinyl chloride articles produced with the inventive ester mixtures feature in particular very good thermostability and are characterized by a low weight loss in the course of thermal ageing in a forced-air oven, and a high HCl stability in the Congo Red test.

The invention will be illustrated in detail with reference to the examples which follow, without any intention that this should bring about a restriction of the invention.

### EXAMPLES

The parts specified are by weight.

Experimental Method

487.5 parts of a polyether polyol formed from trimethylolpropane and propylene oxide with the hydroxyl number 886 mg KOH/g, 631.4 parts of benzoic acid as the aromatic monocarboxylic acid, 500.8 parts of lauric acid as the aliphatic monocarboxylic acid and 188 parts of xylene as the entraining agent were melted under a gentle nitrogen stream in a four-necked flask with stirrer, contact thermometer, water separator, reflux condenser and hotplate with regulator. 2.3 parts of titanium tetra(isopropoxide) were added as the catalyst and the mixture was boiled at 200° C. with stirring for 20 h and then at 220° C. for 2 h. After this time, 132 parts of water had separated out. The volatile constituents were drawn off at 220° C. and 3 mbar within 2 h. After cooling to 120° C., the reaction product was admixed with approx. 30 parts of glass powder and, after stirring for 30 minutes, filtered with suction through a sintered glass suction filter. The liquid was isolated and the acid number determined.

### Examples 1 to 4 and Noninventive Comparative Examples C1 to C5

The inventive compounds 1 to 4 and the noninventive compounds C1 to C5 were prepared by the above method using the starting materials listed in Table 1.

Physical Properties of the Ester Mixtures

The physical data important for ester mixtures (see Table 1) were determined by the following methods:
Viscosity: to DIN 53015 (2001) by means of Höppler falling-ball viscometer
Pour point: to DIN ISO 3016 (1982)

Acid number: to DIN EN ISO 2114 (June 2002)

TABLE 1
Inventive Examples 1 to 4 and noninventive Examples C1 to C5

| | | | | | Hydroxyl | Viscosity | Pour | Dissolution |
|---|---|---|---|---|---|---|---|---|
| | Alcohol | Acid(s) | Molar | Acid number | number | mPa · s | point | temperature |
| Example | (parts) | (parts) | ratio²⁾ | mg KOH/g | mg KOH/g | (23° C.) | ° C. | ° C. |
| 1 | Polyether A¹⁾(390.0) | Benzoic acid (610.6), | 2.5/0.5/1 | 0.3 | 6.7 | 2010 | −10 | 140 |
| | | Lauric acid (200.0) | | | | | | |
| 2 | Polyether A¹⁾(487.5) | Benzoic acid (631.4), | 2/1/1 | 1.0 | 2.9 | 500 | −26 | 150 |
| | | Lauric acid (500.0) | | | | | | |
| 3 | Polyether B¹⁾(390.0) | Benzoic acid (366.4), | 1.5/1.5/1 | 0.3 | 5.6 | 221 | −27 | 163 |
| | | Lauric acid (600.9) | | | | | | |
| 4 | Polyether B²⁾(405.0) | Benzoic acid (557.8), | 3/1/1 | 0.6 | 0.0 | 742 | −13 | 153 |
| | | Lauric acid (304.5) | | | | | | |
| C1 | TMP (134.2) | Lauric acid (620.7) | 0/3/1 | 1.3 | 0.0 | 63 | 12 | >200 |
| C2 | TMP (134.2) | Benzoic acid (378.6) | 3/0/1 | 0.5 | 3.2 | Solid | Solid | Not tested |
| DE 2 318 411 | | | | | | | | |
| C3 | TMP (268.4) | Benzoic acid (495.8), | 2/1/1 | 1.6 | 0.0 | 565⁵⁾ | −26⁵⁾ | 147 |
| | | Lauric acid (406.0) | | | | | | |
| C4 | Polyether C³⁾(301.5) | Lauric acid (400.6) | 0/1/1 | 1.4 | 4.4 | 89 | −12 | >200 |
| C5 | V 250¹⁾ | Benzoic acid (244.2). | 1/2/1 | 0.6 | 5.5 | 131 | −22 | 185 |
| | (390.9) | Lauric acid (800.0) | | | | | | |

¹⁾Polyether A: product of trimethylolpropane and propylene oxide, hydroxyl number 886 mg KOH/g, 3 hydroxyl groups per molecule
²⁾Polyether B: product of pentaerythritol and ethylene oxide, hydroxyl number 837 mg KOH/g, 4 hydroxyl groups per molecule
³⁾Polyether C: product of trimethylolpropane and ethylene oxide, hydroxyl number 362 mg KOH/g, 3 hydroxyl groups per molecule
⁴⁾mole of benzoic acid/mole of fatty acid/mole of alcohol
⁵⁾after storage at room temperature for three weeks, a large amount of a crystalline precipitate forms

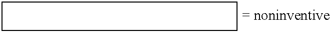

For the handling and processing of ester mixtures as plasticizers, their viscosity and pour point are important characteristic parameters.

Commercial plasticizers are liquids having viscosities between about 10 mPas and more than 10 000 mPas (see, for example, L. Meier: “Weichmacher”, in R. Gächter, H. Müller (Ed.): Taschenbuch der Kunststoffadditive, 3rd edition, p. 383-p. 425, Hanser Verlag, Munich 1990). The examples cited were within this preferred viscosity range.

The pour point indicates the lowest temperature at which a liquid is still free-flowing. The pour points of the examples cited were so low that the substances retain unrestricted free flow at customary room temperatures of above 15° C.

Crystallization Tendency

The processors of soft polyvinyl chloride are equipped for the use of liquid plasticizers. Full or partial crystallization of an originally liquid plasticizer, for example during storage, is undesired, since the redissolution or melting and homogenization constitute additional working steps.

The substance C2 has been proposed in DE 2 318 411 A1 as a plasticizer for hot-melt adhesive preparations. Since C2 is a solid, it is unsuitable for the usual processing to give soft polyvinyl chlorides.

U.S. Pat. No. 3,072,591 B1 describes plasticizers for polyvinyl chloride, based on esters of polymethylolalkanes, for example trimethylolpropane, which contain, in the same molecule, both carboxyl radicals of at least one aromatic carboxylic acid and carboxyl radicals of aliphatic carboxylic acids of at least 6 carbon atoms. However, a complicated two-stage preparation process is needed to prepare these so-called mixed esters (U.S. Pat. No. 3,072,591 B1, column 5, lines 15-17). In order to avoid this undesired complexity, the one-stage process employed in the other examples was used in example C3 to prepare such esters. However, as was found, a large amount of a crystalline precipitate forms in C3 after storage at room temperature for three weeks. The crystallization on storage at 4° C. begins as early as after four days. C3 thus has insufficient storage stability and is therefore unsuitable as a plasticizer.

Surprisingly, and unforeseeably from the prior art, the inventive ester mixtures were notable in that they were liquids at room temperature and did not exhibit any tendency toward crystallization. As the comparison of Example 2 with Comparative Example 3 shows, the inventive esters or ester mixtures based on polyether polyols, in contrast to the noninventive esters or ester mixtures based on polyols, were characterized by a distinctly lower crystallization tendency with identical composition of the acid components. Even after storage at 4° C. for over 21 days, the ester mixture 2 remained clear and fluid.

Dissolution Temperature

The dissolution temperature in polyvinyl chloride is an important characteristic parameter for describing the gelling capacity of a plasticizer. Plasticizers having a dissolution temperature of above 170° C. are not economically viable since their processing demands too much energy. In addition, a dissolution temperature of above 170° C. indicates inadequate compatibility between plasticizer and polyvinyl chloride.

The inventive ester mixtures 1 to 4 had good gelling capacity. The Comparative Examples C1, C4 and C5, whose acid component consists only or predominantly of lauric acid, had dissolution temperatures of above 170° C. and were unsuitable as plasticizers.

Volatility and Thermogravimetry

The volatilities of the inventive ester mixtures 1 to 4 and of the commercial plasticizers dioctyl phthalate (“Vestinol® AH” from Oxeno Olefinchemie GmbH, abbreviaton: DOP) and trioctyl trimellitate (“Eastman TOTM” from Eastman Chemical Company, abbreviation: TOTM) were determined by determining the weight loss in the course of heating of the plasticizer under the conditions specified in Table 2 with the aid of a Brabender H-A-G, E′ moisture tester.

In addition, the abovementioned substances were analyzed by means of thermogravimetry to characterize their volatility. To this end, two different temperature programmes were employed:
1) The samples were kept at 150° C. isothermally for 65 minutes and the remaining amount of ester mixture/plasticizer was weighed. Table 2 reports the residue amount as a percentage of the starting value.
2) The samples were heated at a heating rate of 10° C./min from 25° C. to 400° C. Determined and reported in Table 2 were the particular residue amount (in % of the starting material) which was still present at 245.7° C., and also the temperature at which 50% of the ester mixture/plasticizer had disappeared.

In both of the abovementioned methods, a nitrogen stream of 50 ml/min was passed over the samples. A Mettler Toledo TGA/SDTA 851 e instrument was used.

TABLE 2
Volatility and thermogravimetry

| | Weight loss in | Residue after 65 min | Residue at 245.7° C. | Temperature in ° C. at |
|---|---|---|---|---|
| Example | an oven after | at 150° C. | and heating rate | 50% loss and heating |
| (HAJG) | 6 h at 130° C. | isothermal | 10° C./min | rate 10° C./min |
| 1 | 0.2% | 99.9% | 99.4% | 356.0 |
| 2 | 0.3% | 99.7% | 99.0% | 361.0 |
| 3 | 0.2% | 99.7% | 99.3% | 364.1 |
| 4 | 0.8% | 99.6% | 99.1% | 378.1 |
| DOP | 1.7% | 91.1% | 86.7% | 278.1 |
| TOTM | 0.2% | 99.6% | 98.9% | 338.0 |

The ester mixtrues 1 to 4 featured a lower volatility than the standard plasticizer dioctyl phthalate. The thermogravimetry data reveal that the volatility of the inventive ester mixtures was lower than that of the speciality plasticizer trioctyl trimellitate which is recommended for high-temperature applications owing to its low volatility.

Polyvinyl Chloride Compounds

For further testing, the ester mixtures 1 to 4 and the additives listed in Table 3 were used to produce the soft polyvinyl chloride compounds of the Ca/Zn type (with calcium-zinc stabilizer) and Pb type (with lead stabilizer).

TABLE 3
Composition of the polyvinyl chloride compounds

| | Parts in | |
|---|---|---|
| | Ca/Zn type | Parts in Pb type |
| Constituent | compounds | compounds |
| Polyvinyl chloride (Vinnolit ® H70 DF) | 100 | 100 |
| Plasticizer | 70 | 70 |
| Stabilizer (Crompton Mark ® EZ 735)¹⁾ | 12 | 0 |
| Stabilizer (Interstab ® LGH 6017)² | 0 | 8 |
| Calcium stearate | 1 | 1 |
| Chalk (Omya ® BSH)³ | 30 | 30 |
| Antioxidant (Ciba ® Irganox ® 1010)⁴ | 0.2 | 0.2 |

¹⁾Crompton Mark ® EZ 735 is a Ca/Zn stabilizer.
²Interstab ® LGH 6017 is a lead stabilizer from Akros Chemicals. According to the safety data sheet, it contains approx. 2% lead stearate (CAS No. 1072-35-1), approx. 6% dibasic lead stearate (CAS No. 12578-12-0) and approx. 83% dibasic lead phthalate (CAS No. 57142-78-6).
³Omya ® BSH is a chalk from the Champagne region. The product is coated.
⁴Ciba ® Irganox ® 1010 from SpecialChem S.A. is a phenolic antioxidant having the formula pentaerythtrityl tetrakis(3,5-di-tert-butyl-4-hydroxycinnamate) (CAS No. 6683-19-8).

The components mentioned were first mixed at room temperature and subsequently rolled under the following conditions:

| | |
|---|---|
| Roller: | Servitec Polimix 110 L |
| Temperature: | 160° C. |
| Time: | 10 min |
| Roller speed of front roll: | 20 (rpm) |
| Roller speed of back roll: | 24 (rpm) |
| Thickness of the rolled sheet: | 0.7 mm |

The cooled rolled sheet was then pressed under the following conditions to give films:

| | |
|---|---|
| Press type: | Schwabenthan Polystat 200T |
| Temperature: | 170° C. |
| Time: | 10 min |
| Pressure: | 400 bar |
| Film thickness: | 0.3-1 mm |

Thermal Resistance of the Polyvinyl Chloride Compounds

The thermal resistance of the films was determined by the following test methods:

Storage in a Forced-Air Oven:

Films of size 30×30 mm with a thickness of 1 mm were stored hanging in a forced-air oven at the temperatures and times specified in Table 4. After the storage, the changes in weight were determined and reported in % based on the weight of the film used.

Congo Red Test:

The Congo Red test was carried out to DIN 53381-1 of 1971 using granule obtained from rolled sheets of thickness 0.7 mm. The time after which the colour change of the indicator is visible, occurring as a result of HCl release at 200° C., is listed in Table 4.

TABLE 4
Thermal resistance of the PCV compounds

| | Forced-air | Forced-air | Forced-air | Forced-air | |
|---|---|---|---|---|---|
| | oven | oven | oven | oven | |
| | 7 d/ | 14 d/ | 7 d/ | 14 d/ | Congo |
| Test | 120° C. | 140° C. | 120° C. | 140° C. | Red |
| Compd. type | Ca/Zn | Ca/Zn | Pb | Pb | Pb |
| Plasticizer | Change in | Change in | Change in | Change in | Min. |
| | weight | weight | weight | weight | before |
| | | | | | change |
| 1 | −1.5% | −7.7% | −1.9% | −6.1% | 152 |
| 2 | −1.8% | −6.3% | −2.1% | −7.5% | 180 |
| 3 | −1.4% | −6.3% | −1.5% | −5.5% | 213 |
| 4 | −2.6% | −6.0% | −3.9% | −13.2% | 139 |
| TOTM | −2.4% | −9.7% | −2.3% | −8.1% | 112 |

A high thermal resistance is expressed in a minimum weight loss in the forced-air oven and in a maximum time before change of the Congo Red indicator. The data in Table 4 demonstrate the better thermal stability on average of the inventive ester mixtures in comparison to trioctyl trimellitate (TOTM).

Migration

In order to assess the migration of the inventive ester mixtures from soft polyvinyl chlorides into another polymer, circular test specimens (Ø50 mm) were produced from the above-described Ca/Zn type polyvinyl chloride compound and contacted on both sides with polyethylene film (Atofina Laqtene® LDO 0304), and the contacted test specimens were stored in a drying cabinet at 70° C., weighted down with a 5 kg weight, and a change in weight of the test specimens was monitored over a period of 12 days. Table 5 reproduces the mean values of the changes in weight from a triple determination as % by weight based on the original specimen weights.

TABLE 5
Plasticizer migration

| Ester mixture/ | | | | |
|---|---|---|---|---|
| plasticizer used | 1 day | 2 days | 5 days | 12 days |
| 1 | −0.5% | −0.8% | −0.8% | −1.0% |
| 2 | −0.8% | −1.0% | −1.2% | −1.5% |
| TOTM | −1.2% | −1.5% | −2.1% | −2.6% |

The greater the weight loss measured, the greater the amount of ester mixture/plasticizer which has been transferred into the polyethylene by migration. The data in Table 6 demonstrate the distinctly better migration resistance of the inventive ester mixtures 1 and 2 in comparison to trioctyl trimellitate.

Extraction

In order to assess the extraction of the inventive ester mixtures from soft polyvinyl chloride by liquid media, circular test specimens (Ø 60 mm) were produced from the above-described Ca/Zn type polyvinyl chloride compound and immersed into a Petri dish filled with 50 ml of the media specified in Table 6, and the dishes were stored in a drying cabinet at the temperature specified in Table 6 for 10 days. Afterwards, the change in weight of the cleaned test specimens was determined and is reproduced in Table 6 as a % by weight based on the original sample weights.

TABLE 6
Plasticizer extraction

| | ASTM | ASTM | IRM | IRM | IRM | IRM |
|---|---|---|---|---|---|---|
| Ester mixture/ | oil | oil | 902 | 902 | 903 | 903 |
| plasticizer used | 23° C. | 60° C. | 23° C. | 60° C. | 23° C. | 60° C. |
| 1 | −0.3% | −10.5% | −0.2% | −8.8% | −0.1% | −9.8% |
| 2 | −2.1% | −14.5% | −1.9% | −13.7% | −2.3% | −12.4% |
| TOTM | −6.7% | −18.2% | −4.0% | −15.4% | −8.3% | −14.0% |

The larger the measured weight loss, the greater the amount of plasticizer which has been transferred into the medium by extraction. The data in Table 6 demonstrate the distinctly better extraction resistance of the inventive plasticizers 1 and 2 in comparison to trioctyl trimellitate.

What is claimed is:

1. An ester mixture comprising at least two or more compounds of the general formula (I) 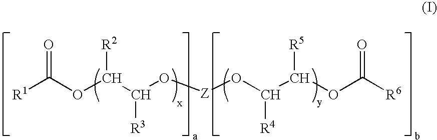
wherein
R¹ is a straight-chain or branched C₅₋ to C₂₁-alkyl radical,
R^{2,} R³, R⁴ and R⁵ are each independently H or C₁₋ to C₄-alkyl radicals,
R⁶ is a C₆- to C₁₄-aryl radical which is optionally substituted by one to three C₁₋ to C₄₋alkyl radicals,
Z is a 3- to 6-valent aliphatic hydrocarbon radical,
x and y are each independently 0 to 6, where x+y has to be >0,
a is 0 to 6, where a has to be >0,
b is 0 to 6 and
a is <or equals b and
the sum of a+b equals the valency of the Z radical.

2. An ester mixture according to claim 1, wherein R¹ derives from aliphatic monocarboxylic acids.

3. A ester mixture according to claim 1, wherein R⁶ derives from aromatic monocarboxylic acids.

4. An ester mixture according to claim 1, wherein Z corresponds to one of the general formulae (II) to (VI) 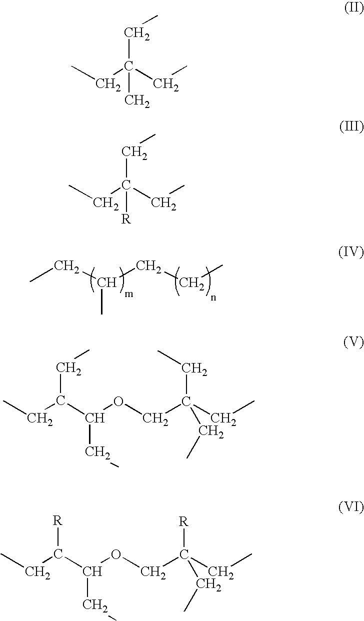
and wherein
R is H or a C₁₋ to C₄-alkyl chain,
m is 1 to 4 and
n is 0 to 3.

5. An ester mixture according to claim 1, wherein Z derives from low molecular weight aliphatic polyalcohols.

6. A process for preparing the ester mixture according to claim 1, wherein
a) one or more polyether polyols having in each case from 3 to 6 hydroxyl groups per molecule are esterified with
b) a mixture of
1) at least 50 mol % of one or more aromatic C₇- to C₁₅-monocarboxylic acids or aromatic C₇- to C₁₅-monocarboxylic acid derivatives and
2) at most 50 mol % of one or more aliphatic C₆- to C₂₂-monocarboxylic acids or aliphatic C₆- to C₂₂-monocarboxylic acid derivatives
where the mixtures 1) and 2) in total have to add up to 100%.

7. A process according to claim 6, wherein the polyether polyols are obtained by ring-opening polymerization or copolymerization of one or more
cyclic aliphatic ethers and
low molecular weight aliphatic polyalcohols having 3 to 6 hydroxyl groups in the molecule.

8. A process according to claim 6, wherein
the aromatic C₇- to C₁₅-monocarboxylic acids or aromatic C₇- to C₁₅-mono-carboxylic acid derivatives may be unsubstituted or C₁- to C₄-alkyl-substituted and/or
the aliphatic C₆- to C₂₂-monocarboxylic acids or aliphatic C₆- to C₂₂-monocarboxylic acid derivatives may be straight-chain or branched, saturated or olefinically unsaturated.

9. A process according to claims 6, wherein the aromatic C₇- to C₁₅-monocarboxylic acids or aromatic C₇- to C₁₅-monocarboxylic acid derivatives are benzoic acid, o-toluic acid, m-toluic acid, p-toluic acid, 4-tert-butylbenzoic acid, 1-naphthoic acid and/or 2-naphthoic acid, or derivatives of these acids or mixtures thereof.

10. A process according to claim 6, wherein the aliphatic C₆- to C₂₂-monocarboxylic acids or aliphatic C₆- to C₂₂-monocarboxylic acid derivatives are hexanoic acid, heptanoic acid, octanoic acid, nonanoic acid, decanoic acid, lauric acid, myristic acid, palmitic acid, stearic acid, oleic acid, arachic acid and/or behenic acid or derivatives of these acids or mixtures thereof.

11. A process for preparing the ester mixture according to claim 6, wherein
polyether polyols prepared from trimethylolpropane and propylene oxide are used;
benzoic acid is used as the aromatic C₇- to C₁₅-monocarboxylic acid; and
lauric acid is used as the aliphatic C₆- to C₂₂-monocarboxylic acid.

12. A method of use of the ester mixtures according to claim 1 as plasticizers for polymers.

13. A method of use of the ester mixtures as plasticizers for polymers according to claim 12, wherein the polymers comprise further additives, such as stabilizers, lubricants, fillers, pigments, flame retardants, light stabilizers, blowing agents, polymeric processing assistants, impact modifiers, optical brighteners, antistats and/or biostabilizers, and also mixtures thereof.

14. A method use of the ester mixtures according to claim 12, wherein the polymers comprise additional plasticizers from the series consisting of monoalkyl esters of benzoic acid, benzoic diesters of mono-, di-, tri- or polyalkylene glycols, dialkyl esters of aliphatic diacids, dialkyl esters of aromatic diacids, trialkyl esters of aromatic triacids, phenyl esters of alkanesulphonic acids, alkyl or aryl esters of phosphoric acid, polyesters of dicarboxylic acids, and also mixtures thereof.
